(19) United States
(12) Patent Application Publication
(10) Pub. No.: US 2007/0233009 A1
(43) Pub. Date: Oct. 4, 2007

(54) Administering Apparatus Having a Priming Function

(76) Inventor: Fritz Kirchhofer, Sumiswald (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/550,659

(22) Filed: Oct. 18, 2006

Related U.S. Application Data
(63) Continuation of application No. PCT/CH05/00215, filed on Apr. 19, 2005.

(30) Foreign Application Priority Data
Apr. 21, 2004 (DE) 10 2004 019 325.8

(51) Int. Cl. A61M 5/00

(52) U.S. Cl. 604/187; 604/207

(57) ABSTRACT

An administering apparatus for administering a fluid product, the apparatus including a casing comprising a reservoir for the product, a driven device which acts on the product contained in the reservoir to deliver the product, a drive device which acts on the driven device, a moveable actuating device coupled to the drive device, the actuating device having at least two different initial positions from which it is moved to actuate the delivery of the fluid product, a restoring device which moves the actuating device, after its movement from one initial position, back to the other initial position, wherein the administering apparatus is formed such that different amounts of delivered fluid are assigned to the movement of the actuating device from the different initial positions.

### CROSS-REFERENCE TO RELATED APPLICATION(S)

This application claims priority to International Application No. PCT/CH2005/000215, filed on Apr. 19, 2005, which claims priority to German Application No. DE 10 2004 019 325.8, filed on Apr. 21, 2004, the contents of both of which are incorporated in their entirety by reference herein.

### BACKGROUND

The present invention relates to devices or apparatus for delivering, dispensing, injecting, administering or infusing substances, and to methods of making and using such devices and apparatus. More particularly, it relates to an administering apparatus for administering a fluid product, including for medical, including veterinary, therapeutic, diagnostic, pharmaceutical and/or cosmetic applications. In some preferred embodiments, injection apparatus, such as injection pens, and also inhalation apparatus are examples of administering apparatus.

In the aforementioned uses or applications of administering or injection devices, it is often important for a very particular product dosage, i.e. a defined product amount, to be administered. One source of uncertainty in this respect is if a part of the apparatus which contains the product, in particular a medical product or medicine such as insulin or heparin, is not completely filled with the product but contains air. If not vented, the air or more generally a gas will be administered together with the product. In the case of subcutaneous administering for example, this would not lead to health complications, but the correct product dosage would still not be administered, but rather a dosage reduced by the air content. In other types of administering, however, impairment to health can in addition also occur if air is administered together with the product. Air can be present in the product-guiding part of the administering apparatus not only when product is administered from a reservoir for the first time, but also again after administering for the first time. A liquid product in an injection cannula can, for example, vaporise from the cannula tip and the cannula can dry up. If the product cannot subsequently flow or not flow sufficiently rapidly from the reservoir side due to the prevailing pressure ratios, a volume of air formed in the injection cannula from the tip. If product is later administered again, without venting beforehand, then the administered product dosage is reduced in accordance with this volume of air.

Administering apparatus of the general type of the present invention are known from WO 97/36625, DE 199 00 792 C1 and U.S. Pat. No. 6,228,067 B1. The administering apparatus described in these documents are injection pens which allow the product dosage which is to be administered by injection to be selected. The user requires some experience and practice in order to prime the known apparatus, as venting is generally referred to. The user pushes product “by instinct” through the injection needle of the respective apparatus until he confirms product escaping at the needle tip by a visual check, wherein it is possible for an unnecessarily large amount of product to be delivered for the purpose of priming. Moreover, a visual check is often difficult for people with impaired vision and causes an increased risk of the priming process being incorrectly performed.

An injection apparatus having a priming function is known from WO 2004/007000 A1, wherein priming by instinct is avoided and it is possible to change between a priming function and an active agent function by means of a movement transverse to the longitudinal axis of the administering apparatus. This change is made by a rotational movement. This movement must be performed exactly in order to ensure a correspondingly correct function of the administering apparatus. This can cause problems if the user's fine motor system is impaired.

### SUMMARY

It is an object of the present invention to provide an administering apparatus having a priming function, which is simple to operate. Since it is user-friendly, this increases the certainty that a particular product dosage known to the user is also actually administered.

In one embodiment, the present invention comprises an apparatus for delivering a fluid product, the apparatus comprising a driven device which acts on the product to deliver the product, a drive device which acts on the driven device, a moveable actuating device coupled to the drive device, the actuating device having two different initial positions from which it is moveable to actuate the delivery of the fluid product, a restoring device which moves the actuating device, after its movement from one initial position, back to the other initial position, wherein the apparatus is formed such that different amounts of delivered fluid are assigned to or correlated with the movement of the actuating device from the different initial positions.

The user-friendliness can be increased if, in order to actuate the administering apparatus, a movement has to be performed which is already known to the user from the everyday use of other objects. A ballpoint pen is one example of a generally common object. Actuating the push-button of a ballpoint pen is simple to perform and there is a definite change between a writing and non-writing state.

In one embodiment, an administering apparatus for administering a fluid product, such as the invention relates to, comprises a casing, a driven device, a drive device, an actuating device and a restoring device. The casing comprises a reservoir for the fluid product. It is intended that the term “fluid” relates to both gases and liquids. The driven device acts on the product contained in the reservoir, in order to deliver the product. The term “deliver” is to be interpreted in a general sense and can refer both to delivering the fluid product for priming and to delivering the fluid product to deploy the active agent in a targeted manner. The drive device acts on the driven device. It therefore ensures that the driven device, which acts on the product contained in the reservoir, delivers the product. The drive device provides for the transfer of energy onto the driven device, wherein the drive itself can for example be mechanical or electric. The actuating device is movable and coupled to the drive device. Actuating the actuating device, for example, according to the push-button principle, thus activates the mechanism of the drive device. The actuating device is movable and can, for example, be shifted, compressed or rotated. The actuating device can be formed in one or more parts. If formed in multiple parts, the individual components can be produced from the same material or from different materials. In some preferred embodiments, the coupling between the actuating device and the drive device is a mechanical coupling, but other types of coupling are also conceivable.

The actuating device further comprises at least two different initial positions, from which the actuating device can be set in motion to deliver the fluid product. These different initial positions can be defined by different initial locations. Two initial positions are, for example, situated on different sides with respect to the central longitudinal axis of the administering apparatus; they can also be different distances away from the ends of the administering apparatus, such as for example an injection needle, with respect to their position as projected onto the longitudinal axis. The different initial positions can also be defined on the basis of a deformation of the actuating device; they can for example result from compressing the actuating device. The different initial positions of the actuating device may be defined by a respectively different orientation of a part of the actuating device with respect to the longitudinal axis of the administering apparatus. This orientation is, for example, achieved by rotating or tilting a component of the actuating device or by rotating or tilting in combination with shifting. The restoring device of the administering apparatus moves the actuating device, after its movement from one initial position, back into the other or another initial position, in some preferred embodiments, automatically, for example by using an elastic means (e.g., a spring or rubber). The restoring movement of the actuating device which thus occurs can include a rotational or tilting movement and/or a translational movement. The administering apparatus is formed such that different amounts of delivered fluid are assigned to the movement of the actuating device from the different initial positions. The movement of the actuating device from the different initial positions thus initiates the delivery of the fluid, the respective initial position defining the amount of the product to be delivered. The fluid can be delivered at the same time as the movement begins, or can also begin after a time delay. In one preferred embodiment of the administering apparatus, the product is delivered at the same time as the movement of the actuating device and ends at the moment in which the movement of the actuating device ends.

In some preferred embodiments, if two initial positions are formed, then one amount of the fluid product to be delivered is a priming amount and the other amount of the fluid product to be delivered is an active agent amount used to deploy the active agent in doses, wherein the priming amount is advantageously significantly smaller than the active agent amount, so as not to waste the product.

In some preferred embodiments, actuating the actuating device consists of a movement of an element of the actuating device along the longitudinal axis of the administering apparatus. Such actuating can, for example, consist of pushing a push-button in the direction of said longitudinal axis. In principle, however, a rotational movement is also possible for actuating the actuating device, or a combination of a translational movement and a rotational movement.

In accordance with a preferred embodiment of the present invention, the restoring device comprises an elastic element. This elastic element can for example be deformed. In some preferred embodiments, the elastic element is a spring system. It is also conceivable in principle to use an inelastic element, which is moved—mechanically or electrically driven—for the purpose of restoring, instead of an elastic element.

In accordance with another preferred embodiment of the invention, the reverse movement of the actuating device ends in two different initial positions. A reverse movement is understood to means a movement directed counter to the original movement. If, for example, the movement of the actuating device during product delivery includes a movement portion in the direction of the longitudinal axis of the administering apparatus towards the product outlet opening, then in the reverse movement there is a movement portion along the longitudinal axis of the administering apparatus away from the product outlet opening. These purely translational movements can, of course, also be overlaid with rotational or tilting movements. These rotational or tilting movements can occur or extend congruently during the forward movement and reverse movement of the actuating device, but, in some preferred embodiments, are different.

In some preferred embodiments, the administering apparatus comprises an end position which delimits the movement of the actuating device in order to terminate delivery of the fluid product, wherein product delivery of the different amounts therefore begins in different initial positions, but the end position for terminating delivery is the same in both cases. If, for example, two initial positions are situated on different sides with respect to the central longitudinal axis of the administering apparatus and their positions as projected onto the longitudinal axis are different distances away from the ends of the administering apparatus, and if the movement of the actuating device from the different initial positions is then directly and continuously converted into product delivery up until a common end position, then it is immediately clear that different product delivery amounts result from the common end position. In principle, however, multiple end positions are also conceivable which are assigned to the respective initial positions and define the respective product amount which is delivered. In some preferred embodiments, however, a single end position is used, which simplifies the manufacture of the administering apparatus.

In some preferred embodiments, the aforementioned end position is formed by a stopper, wherein a movable part of the actuating device is prevented from moving further when it contacts the stopper.

In some embodiments, the actuating device is coupled to the drive device by a coupling piece. Said coupling piece can be formed in one or more parts. The coupling piece is movable and can be shifted and/or rotated in one or more directions. The coupling piece can be formed rigid or elastic.

In a preferred embodiment, the administering apparatus comprises two stoppers which are alternately connected to the coupling piece. The position of the coupling piece with respect to the two stoppers defines the two initial positions of a device in accordance with the present invention. The connection between the coupling piece and a stopper can merely be contact; in some preferred embodiments, however, the two stoppers are formed as locking stoppers which lock or hook onto the coupling piece, with a clearance. Other forms of a connection are in principle also possible, for example magnetic fixing. An alternating connection is understood to mean a connection which is alternately connected first to one stopper, then to the other. The coupling piece is advantageously never connected to both locking stoppers at the same time, though in principle this could be the case in transition between the connection between the coupling piece and one locking stopper and the connection between the coupling piece and the other locking stopper.

In a preferred embodiment of the present invention, one of the aforementioned stoppers is a priming stopper and the other is an active agent stopper. Priming is initiated from the priming stopper, and a defined administering of an active agent is triggered from the active agent stopper by a movement of the actuating device.

In some embodiments, the coupling piece consists of a transfer piece and a sliding piece which are connected to each other. Both the transfer piece and the sliding piece can be formed in one or more parts. The transfer piece and the sliding piece are engaged with each other, wherein a clearance exists between the transfer piece and the sliding piece. To this end, the transfer piece comprises a cavity with which the sliding piece engages.

In some embodiments, the transfer piece may be formed with two limbs. Being formed with two limbs can, for example, be understood to mean V-shaped or U-shaped transfer pieces. The two limbs of the transfer piece can be formed in different sizes or in the same size, they can exhibit a symmetry with respect to each other, or they can be shaped completely differently. In the sense of the present invention, being formed with two limbs can also be defined by the functionality of the limbs; for example, one limb comes into contact with a priming stopper, the other with an active agent stopper.

In some preferred embodiments, if the administering apparatus in accordance with the present invention is provided with a transfer piece and two stoppers, e.g., locking stoppers, then the transfer piece comprises two angled sections which hook onto the two stoppers. If a transfer piece has two limbs, the two limbs can each comprise an angled section. The small angled sections serve to better hook the transfer piece onto the two stoppers.

In a preferred embodiment of the present invention, the transfer piece can be tilted and/or rotated about the locking stoppers. A tilting movement is to be understood to mean a rotational movement about a fulcrum. This movement can be overlaid with a translational movement as applicable. In the present invention, the tilting or rotational movement of the transfer piece occurs in the following situation: one limb of the transfer piece is slightly angled at its end and hooked via this angled end onto locking stopper; if the transfer piece is then set in motion by actuating the actuating device, then this can for example bring it under pressure or tension, wherein it initially remains connected to the locking stoppers and gives way to the acting force by tilting or rotating. The transfer piece then detaches from one locking stopper and is moved in a reverse movement to the second locking stopper which the other limb is hooked onto.

In a preferred embodiment, the sliding piece of the administering apparatus can be shifted in a direction orthogonal to the longitudinal axis of the administering apparatus. This movement can be superimposed with a movement in the direction of the longitudinal axis of the administering apparatus. In some preferred embodiments, the sliding piece can be shifted orthogonal to the longitudinal axis of the administering apparatus from one casing wall of the administering apparatus to the casing wall of the administering apparatus lying opposite with respect to the longitudinal axis of the administering apparatus. When in contact with the casing walls, the sliding piece comes to rest there in a fitting shape. In a preferred embodiment, the movement of the sliding piece in the direction of the longitudinal axis of the administering apparatus is directly transferred onto the movement of the drive device and/or driven device of the administering apparatus and therefore onto the delivery of the product.

In a preferred embodiment, the administering apparatus comprises a symmetrical transfer piece with two limbs, which is engaged, with a clearance, with a sliding piece which can be shifted. In a first initial position, the axis of symmetry of the transfer piece is inclined with respect to the longitudinal axis of the administering apparatus and hooked onto a first locking stopper via a limb. The actuating device is actuated by pushing a button and transferring said pressure force onto the transfer piece along the longitudinal axis of the administering apparatus. Since the position of the axis of symmetry of the transfer piece is inclined with respect to the longitudinal axis of the administering apparatus and since the transfer piece is engaged with the sliding piece, the transfer piece exerts a lever effect on the sliding piece. Under the increasing pressure from the actuating device, the transfer piece detaches from the first locking stopper and moves the sliding piece in the direction of the second locking stopper along the transverse axis of the administering apparatus. Once the user lets go of the actuating device, the restoring device automatically moves the transfer piece into the second initial position, i.e. in its reverse movement, the transfer piece then automatically hooks onto a second locking stopper via its second limb. Due to this hooking and the restoring force acting along the longitudinal axis of the administering apparatus, the orientation of the axis of symmetry of the transfer piece is altered with respect to the longitudinal axis of the administering apparatus; in the second initial position, the axis of symmetry is inclined with respect to the longitudinal axis in the other direction to that in the first initial position. When actuating the administering apparatus out of the second initial position, the processes proceed analogously and automatically end in the first initial position.

In one preferred embodiment of the present invention, the administering apparatus comprises a dosing member using which the active agent amount of the fluid product to be administered is set. It is thus for example possible to combine a consistent, small priming amount with a settable, in particular alternating, active agent amount of the fluid product which is actually to be administered.

In accordance with a preferred embodiment, the administering apparatus comprises a driven device comprising a piston which is moved in a direction along the longitudinal axis of the administering apparatus when product is administered and is prevented from moving in the opposite direction by a blocking device. This provides additional certainty that air is prevented from undesirably entering the administering apparatus. The blocking device can for example consist of a row of teeth along which a movement is only possible in one direction. The blocking device can alternatively be made of a specially bent plastic which prevents the piston from slipping back.

In accordance with another aspect, the present invention relates to a method for using the administering apparatus. In one embodiment, this method comprises the following steps: actuating the actuating device for a first time in a first initial position; delivering a first amount of a fluid product; restoring the actuating device to a second initial position; actuating the actuating device for a second time in the second initial position; delivering a second amount of a fluid product; restoring the actuating device to the first initial position. The method therefore begins in a first initial position in which the actuating device or at least a component of the actuating device is situated. Actuating the actuating device triggers the delivery of a first amount of a fluid product, at the same time or after a delay. Actuating the actuating device consists of pushing an actuating button such as is already known to the users of administering apparatus from the use of ballpoint pens. The push-button can be pushed in up to a certain extent, then abuts a barrier; once the user lets go of the push-button, the push-button undergoes a restoring movement. The actuating device is restored to a second initial position. The second initial position can, for example, differ from the first initial position in that their positions are different distances away from the product outlet opening of the administering apparatus along the longitudinal axis of the administering apparatus, or in that the actuating device assumes different positions relative to the casing. Precisely these relationships are encountered in the example of a conventional ballpoint pen. Depending on whether or not it is possible to write with the ballpoint pen, the push-button of the ballpoint pen is situated a different distance away from the nib of the ballpoint pen and/or the writing nib of the ballpoint pen protrudes out of it or is hidden within it.

In another embodiment of the present invention, the two initial positions also differ, for example, by a different rotational angle with respect to the longitudinal axis of the administering apparatus. Actuating the actuating device for the second time differs from actuating the actuating device for first second time in that it is triggered in the second initial position. It initiates a process for delivering a second amount of a fluid product. This delivery can be made at the same time as the actuating device is actuated, or also after a time delay. After a second amount of a fluid product has been delivered, the actuating device is restored again to the first initial position. In some embodiments, a ballpoint pen type mechanism is used in the present invention. Actuating in the first initial position delivers the first amount and actuating in the second initial position delivers the second amount.

The method described is performed cyclically; correspondingly, it can be repeated any number of times. The method as a whole can likewise be understood analogously to actuating a ballpoint pen. With a ballpoint pen, it is also possible to change between a writing and non-writing state of the ballpoint pen by repeatedly actuating the push-button on the ballpoint pen.

### BRIEF DESCRIPTION OF THE DRAWINGS

FIG. 1 depicts an embodiment of the present invention comprising an injection apparatus in a longitudinal section;

FIGS. 2A-2E depict the actuating section of the embodiment depicted in FIG. 1.

### DETAILED DESCRIPTION

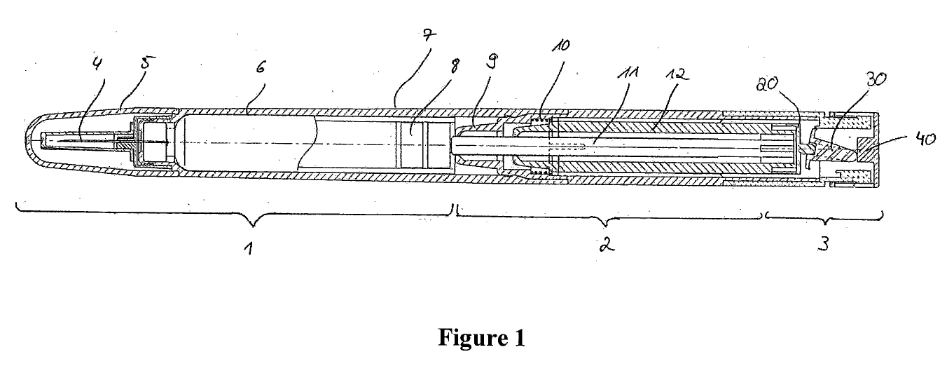

FIG. 1 shows an exemplary injection apparatus in accordance with the present invention in a longitudinal section. In the example shown, the injection apparatus may be divided into three main sections: an injection section 1, a drive section 2 and an actuating section 3. This division should not be interpreted too strictly and is merely one of a number of possibilities.

The injection section 1 contains a reservoir 6 in a casing 7, the fluid product being situated in the reservoir 6. The fluid product passes from the reservoir 6 into an injection needle 4 using which the active agent is, for example, injected into a human body. If the injection apparatus is not currently being used, then the needle 4 is protected by a protective cap.

The fluid product is pushed out of the injection apparatus with the aid of a piston 8. In the present example, the piston 8 is moved by means of a piston rod 11. The piston 8 and the piston rod 11 can only be moved in the direction of the outlet opening of the injection apparatus; a blocking device 9 provides for this.

The actuating section 3 contains the components of the actuating device. In the present exemplary case, these are the actuating button 40 of the actuating device, a V-shaped transfer piece 30 and a sliding part 20 which can be shifted. The transfer piece 30 and the sliding part 20 together form a coupling piece. A spring system 10, which functionally can likewise be classed as the actuating device, is situated outside of the actuating section 3 in the drive section 2. The transfer piece 30 is shifted and rotated by pressing-in the actuating button 40. In some preferred embodiments, the transfer piece 30 is engaged, with a clearance, with the sliding part 20, which is moved with it in accordance with the movement of the transfer piece 30. The sliding part 20 acts on the slaving part 12, which in turn—during movement towards the outlet opening—slaves the drive device or piston rod 11 and thus moves the piston 8 in the direction of the outlet opening of the injection apparatus, such that product is pushed out of the injection apparatus. If, once the product has been delivered, the sliding part 20 is moved back in the reverse direction away from the outlet opening of the injection apparatus due to the force of the spring system 10, then the piston rod 11 does not take part in this movement. The piston rod 11 is prevented from doing so by the blocking device 9. The slaving part 12 does, however, take part in this movement and assumes a new position relative to the piston rod 11, forming a lock. During repeated product delivery processes, the piston rod 11 therefore moves further and further away from the sliding piece 20 or the end of the injection apparatus at which the actuating device 3 is situated, while the slaving part 12 remains in contact with it.

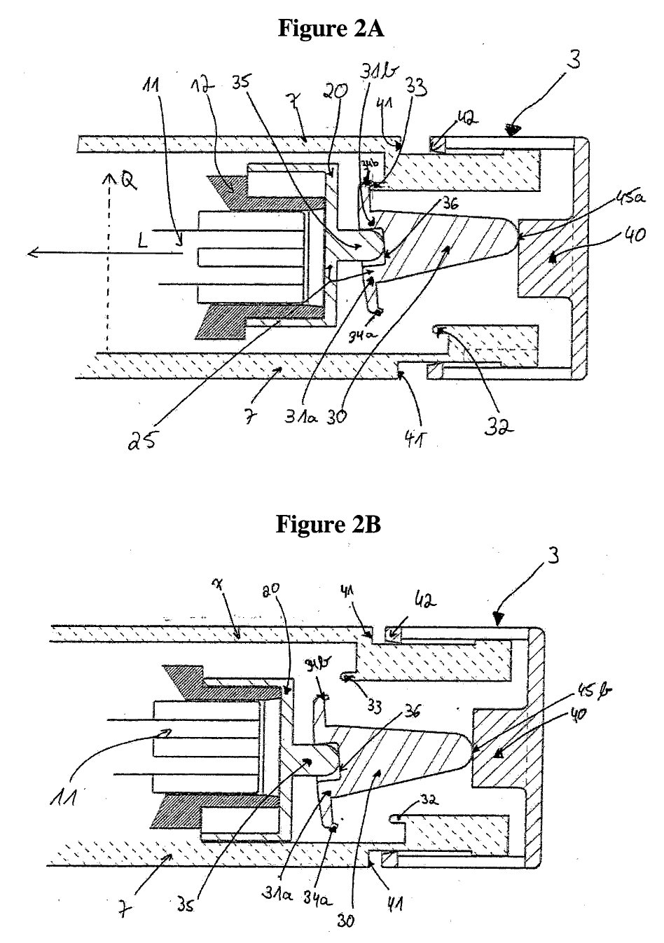

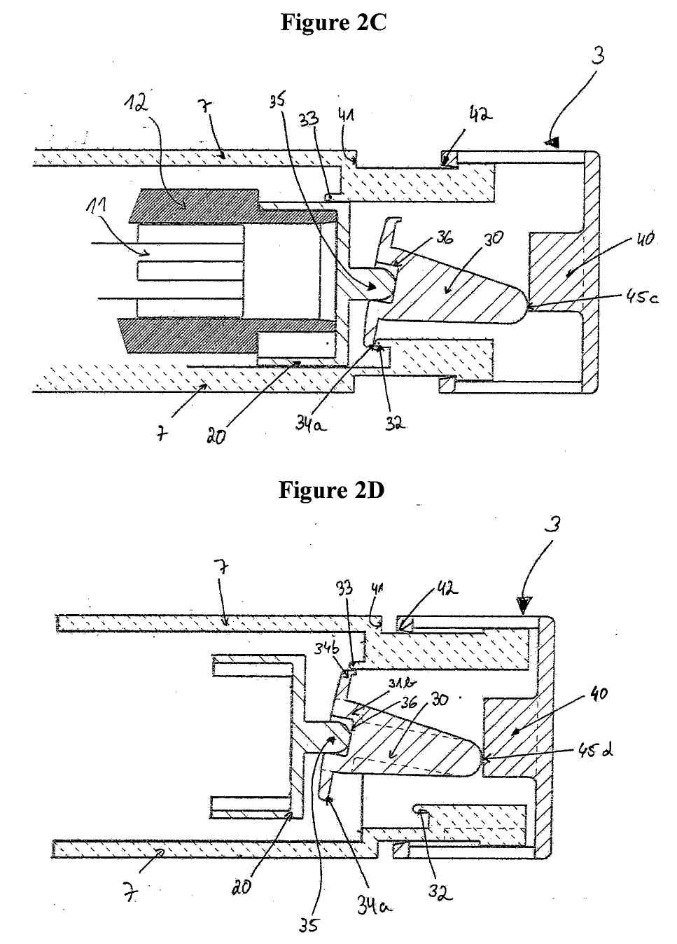

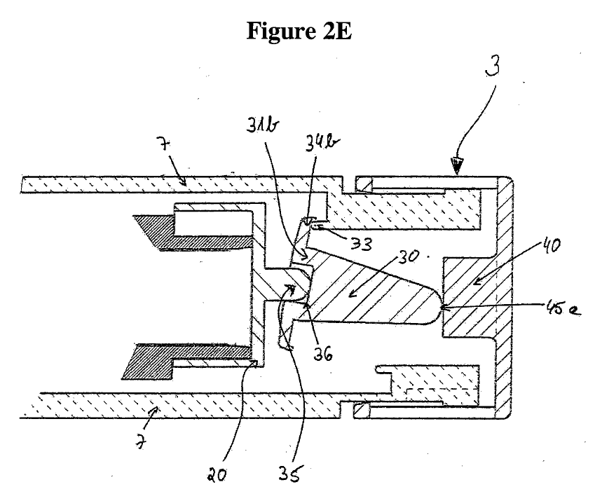

FIGS. 2A-2E show detailed representations of an embodiment of an actuating section 3 in an enlargement. FIGS. 2A to 2E are intended to illustrate, by example, the components and functionality of an actuating device in accordance with the present invention.

FIG. 2A shows the actuating section 3 directly before the injection apparatus is primed. The actuating section 3 is substantially enclosed by a casing 7. The rear part, however, is directly formed by an actuating button 40 which exhibits a cap-like embodiment and is plugged onto the casing 7. The actuating button 40 can be shifted along the longitudinal axis L with respect to the casing 7 up to an end position 41 formed as a stopper. The central region of the actuating button with respect to the longitudinal axis L is formed as an elevation. The transfer piece 30 abuts this elevation in a position 45a. The transfer piece 30 is shaped in an approximate V shape and is slightly inclined with respect to the longitudinal axis L. It comprises a cavity 36 and is partitioned into two—in this case, symmetrical—limbs 31a and 31b. Small angled sections or barbs 34a and 34b are situated at the end of each of these limbs 31a and 31b and can fulfil the function of a hook, hook-like connection or coupling. One limb 31b of the transfer piece 30 is in contact with a locking stopper 33; it is made of the same material and component as the casing 7 itself. The locking stopper 33 is formed at a certain distance longitudinally from the wall of the injection apparatus with respect to the longitudinal axis L. In some embodiments, the limb 31b is preferably hooked onto or coupled to the locking stopper 33 with a certain clearance by means of the angled section 34b.

The angled section 34b can be moved or tilted rotationally about the locking stopper 33. In some preferred embodiments, the other side of the transfer piece 30 comprising the other limb 31a and the angled section 34a is situated out of any contact before the priming process is begun.

A protrusion 35 of a sliding piece 20 engages, with a clearance, with the cavity 36. The sliding piece can be moved along the axis L and orthogonally to the axis L along the axis Q. The sliding piece 20 is in indirect contact with the piston rod 11 which can only be moved in the direction of the axis L. Before the priming process is begun, the sliding piece 20 is shifted slightly upwards with respect to the central L axis in the direction of the Q axis. The continuation 35 of the sliding piece 20 correspondingly abuts in the upper region of the transfer piece 30.

If the priming process is then performed, then the actuating button 40 is pushed in, against the spring force of the restoring device 10, along the axis L until the lateral enclosures 42 of actuating button 40 reach the stopper 41. The movement of the actuating button also moves the transfer piece 30. It detaches from the locking stopper 33 in a rotational movement. The transfer piece 30 is thus tilted or inclined. It acts as a lever on the sliding piece 20.

FIG. 2B shows the actuating section 3 shortly before the priming process is performed. The actuating button 40 has just been relaxed again and is moved backwards again by the restoring device 10, mediated by the sliding piece 20 and the transfer piece 30. A small gap has already been created again between the end stopper 41 and the lateral enclosure 42 of the actuating button 40. The bearing point 45b of the transfer piece 30 has been displaced downwards on the actuating button 40. The sliding piece 20 has moved downwards along the Q axis and now lies on the bottom of the casing 7. The transfer piece 30 has correspondingly also moved downwards. Preferably, neither of the two limbs 31a and 31b of the transfer piece 30 is in contact via its angled ends 34a and 34b with a locking stopper 32 or 33 at this point in time.

FIG. 2C shows the actuating section 3 at a slightly later point in time, after the restoring movement has been completed by the transfer piece 30 or its angled section 34a hooking-in on the locking stopper 32. The spring system 10 or sliding piece 20 pushes the transfer piece 30 backwards as far as possible. This inclines the transfer piece 30 in the opposite direction to that in FIG. 2A with respect to the longitudinal axis L of the injection apparatus. The bearing point 45c of the transfer piece 30 now lies in the lower region of the elevation of the actuating button 40. The sliding piece 20 is engaged, with a clearance, with the transfer piece 30 in the lower part of the cavity 36 of the transfer piece 30. In the position in FIG. 2C, the upper region of the sliding piece 20 has slipped or retracted below the locking stopper 33. In this position, the injection apparatus shown in FIG. 2C is ready for delivering the active agent amount to be injected. In this position, the guiding point 42 of the actuating button 40 is at its furthest away from the end stopper 41, hence a large active agent amount can subsequently be delivered.

FIG. 2D shows the administering apparatus shortly before completing the process of delivering the active agent amount to be injected. The guiding point 42 of the actuating button 40 has already moved a fair way towards the end stopper 41. The angled section 34a of the connecting piece 30 has already detached again from the rear locking stopper 32. The transfer piece 30 is beginning to rotate again and to move forwards in the direction of the L axis; the bearing point 45d on the actuating button 40 is moving upwards again. The sliding piece 20 passively follows the transfer piece 30. Shortly before the product delivery is completed, the transfer piece 30 comes into contact with the locking stopper 33 via its angled part 34b below the locking stopper 33 and slides along it. If the actuating button 40 is completely pushed in, then the angled section 34b slides past the locking stopper 33 and can hook onto it during the following reverse movement. For reasons of simplicity, the piston rod 11 has not been shown in FIG. 2D and is also omitted in FIG. 2E described below.

FIG. 2E shows the injection apparatus shortly before dosing the priming amount. The limb 31b of the transfer piece 30 has already hooked around the priming locking stopper 33 via its angled continuation 34b. In the subsequent progression of this second restoring movement, the actuating button 40 is also moved slightly to the right, i.e. away from the opening of the injection apparatus. Since the transfer piece 30 is hooked onto the priming locking stopper 33, the transfer piece 30 undergoes a rotation about the priming locking stopper 33 during this movement; as a whole, the transfer piece 30 is also rotated and/or tilted slightly further. During this movement, the axis of symmetry of the transfer piece 30 moves beyond the central longitudinal axis L of the injection apparatus and is finally—as already shown in FIG. 2A—orientated differently to the longitudinal axis L with respect to the pitch of the axis of symmetry.

The process described in FIGS. 2A-2E can be preformed cyclically and correspondingly repeatedly. The product amount to be delivered in each process step is decided by the respective locking position 32 or 33 from which the delivery process is begun. The path to be travelled by the actuating button 40 from the guiding point 42 up to the common end stopper 41 also differs in length in accordance with the locking positions 32 or 33 assumed. These two different lengths are directly related to the two different product amounts which are delivered as a priming amount in one case and as an active agent amount in the other.

Embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An administering apparatus for administering a fluid product, comprising:
a) a casing comprising a reservoir for the product;
b) a driven device which is formed to deliver the product when it is situated in the reservoir;
c) a drive device which acts on the driven device;
d) an actuating device, which
is movable,
is coupled to the drive device, and
comprises at least two different initial positions, from which the actuating device is moveable to actuate delivery of the product; and
e) a restoring device which moves the actuating device, after its movement from one initial position, back into the other initial position; wherein
f) the administering apparatus is formed such that different amounts of product to be delivered are assigned to the movement of the actuating device from the different initial positions.

2. The administering apparatus in accordance with claim 1, wherein one amount of the delivered fluid product is a priming amount and the other amount of the delivered fluid product is an active agent amount used to deploy the active agent in doses.

3. The administering apparatus in accordance with claim 1, wherein actuating the actuating device comprises moving an element of the actuating device along the longitudinal axis of the administering apparatus.

4. The administering apparatus in accordance with claim 1, wherein the restoring device comprises an elastic element.

5. The administering apparatus in accordance with claim 1, wherein a restoring movement of the actuating device ends in one of the two different initial positions.

6. The administering apparatus in accordance with claim 1, wherein the administering apparatus comprises an end position which delimits the movement of the actuating device to terminate delivery of the fluid product.

7. The administering apparatus in accordance with claim 6, wherein the end position is defined by a stopper.

8. The administering apparatus in accordance with claim 1, further comprising a coupling piece which couples the actuating device to the drive device.

9. The administering apparatus in accordance with claim 8, further comprising two stoppers which are alternately connected to the coupling piece.

10. The administering apparatus in accordance with claim 9, wherein one of the stoppers is a priming stopper and the other is an active agent stopper.

11. The administering apparatus in accordance with any one of claim 8, wherein the coupling piece comprises a transfer piece and a sliding piece.

12. The administering apparatus in accordance with claim 11, wherein the transfer piece is formed with two limbs.

13. The administering apparatus in accordance with claim 11, wherein the transfer piece comprises two angled sections which hook onto the two stoppers.

14. The administering apparatus in accordance with claim 13, wherein the transfer piece is tiltable and/or rotatable about the stoppers.

15. The administering apparatus in accordance with claims 11, wherein the sliding piece is shiftable in a direction orthogonal to the longitudinal axis of the administering apparatus.

16. The administering apparatus in accordance with claim 9, wherein the stoppers are locking stoppers.

17. The administering apparatus in accordance with claim 1, further comprising a dosing member using which the active agent amount of the fluid product to be administered is set.

18. The administering apparatus in accordance with claim 1, wherein the driven device comprises a piston moveable in a direction along the longitudinal axis of the administering apparatus when product is administered and prevented from moving in the opposite direction by a blocking device.

19. An apparatus for delivering a fluid product, the apparatus comprising a driven device which acts on the product to deliver the product, a drive device which acts on the driven device, a moveable actuating device coupled to the drive device, the actuating device having two different initial positions from which it is moveable to actuate the delivery of the fluid product, a restoring device which moves the actuating device, after a movement from one initial position, back to the other initial position, wherein different amounts of delivered fluid are correlated with the movement of the actuating device from the different initial positions.

20. A method for using an administering apparatus, comprising the steps of:
a) actuating an actuating device for a first time in a first initial position;
b) delivering a first amount of a fluid product;
c) restoring the actuating device to a second initial position;
d) actuating the actuating device for a second time while the actuating device is in the second initial position;
e) delivering a second amount of a fluid product; and
f) restoring the actuating device to the first initial position.

21. The method for using the administering apparatus in accordance with claim 20, wherein the steps of the method are repeated cyclically.
